SEQUENCE LISTING (207 sequences)

SEQ ID NO: 1 - DNA, 19 bp, synthetic construct
  source 1..19: mol_type = other DNA; note = ZmGBSSIZmGBSSI-asgRNA1
ggccccctgatctcgcgcg

SEQ ID NO: 2 - DNA, 19 bp, synthetic construct
  source 1..19: mol_type = other DNA; note = sgRNA2
gcaatataaaattaccgat

SEQ ID NO: 3 - DNA, 19 bp, synthetic construct
  source 1..19: mol_type = other DNA; note = ZmGBSSI-bsgRNA1
caccacgcgcgagatcagg

SEQ ID NO: 4 - DNA, 19 bp, synthetic construct
  source 1..19: mol_type = other DNA; note = sgRNA2
ataagaacaccgaacagca

SEQ ID NO: 5 - DNA, 19 bp, synthetic construct
  source 1..19: mol_type = other DNA; note = ZmSH2SH2-asgRNA1
acaaaacaagaacctacaa

SEQ ID NO: 6 - DNA, 19 bp, synthetic construct
  source 1..19: mol_type = other DNA; note = sgRNA2
tacatactagcagcacaaa

SEQ ID NO: 7 - DNA, 19 bp, synthetic construct
  source 1..19: mol_type = other DNA; note = SH2-bsgRNA1
ggatcgtgtaaccttggta

SEQ ID NO: 8 - DNA, 19 bp, synthetic construct
  source 1..19: mol_type = other DNA; note = sgRNA2
gttgatccaaagcaggtaa

SEQ ID NO: 9 - DNA, 19 bp, synthetic construct
  source 1..19: mol_type = other DNA; note = ZmAGPLS3AGPLS3-asgRNA1
tttcaaagcagcaaccaca

SEQ ID NO: 10 - DNA, 19 bp, synthetic construct
  source 1..19: mol_type = other DNA; note = sgRNA2
tacatgtagagggagagat

SEQ ID NO: 11 - DNA, 19 bp, synthetic construct
  source 1..19: mol_type = other DNA; note = AGPLS3-bsgRNA1
agcacctgcaacacgccaa

SEQ ID NO: 12 - DNA, 19 bp, synthetic construct
  source 1..19: mol_type = other DNA; note = sgRNA2
acttggcacatgagatgca

SEQ ID NO: 13 - DNA, 19 bp, synthetic construct
  source 1..19: mol_type = other DNA; note = Bt2Bt2-asgRNA1
tgtgtccttcaatgttgta

SEQ ID NO: 14 - DNA, 19 bp, synthetic construct
  source 1..19: mol_type = other DNA; note = sgRNA2
tctaacacataggatgcgg

SEQ ID NO: 15 - DNA, 19 bp, synthetic construct
  source 1..19: mol_type = other DNA; note = Bt2-bsgRNA1
tattcgtcaggcacaaaag

SEQ ID NO: 16 - DNA, 19 bp, synthetic construct
  source 1..19: mol_type = other DNA; note = sgRNA2
caatccactccattctaca

SEQ ID NO: 17 - DNA, 19 bp, synthetic construct
  source 1..19: mol_type = other DNA; note = ZmDOF36DOF36-asgRNA1
caccgaccagatgatgcga

SEQ ID NO: 18 - DNA, 19 bp, synthetic construct
  source 1..19: mol_type = other DNA; note = sgRNA2
ttaacaaagaaataaactg

SEQ ID NO: 19 - DNA, 19 bp, synthetic construct
  source 1..19: mol_type = other DNA; note = DOF36-bsgRNA1
ctccctcgcatcatctggt

SEQ ID NO: 20 - DNA, 19 bp, synthetic construct
  source 1..19: mol_type = other DNA; note = sgRNA2
catcaagcagatataacgc

SEQ ID NO: 21 - DNA, 19 bp, synthetic construct
  source 1..19: mol_type = other DNA; note = ZmTPS9TPS9-asgRNA1
cggagaacctgaatccgaa

SEQ ID NO: 22 - DNA, 19 bp, synthetic construct
  source 1..19: mol_type = other DNA; note = sgRNA2
tatcttgacagtcacggtg

SEQ ID NO: 23 - DNA, 19 bp, synthetic construct
  source 1..19: mol_type = other DNA; note = ZmAuxRP1AuxRP1-asgRNA1
atcacctgaacatggtgcg

SEQ ID NO: 24 - DNA, 19 bp, synthetic construct
  source 1..19: mol_type = other DNA; note = sgRNA2
cagagccaccctcgccggt

SEQ ID NO: 25 - DNA, 19 bp, synthetic construct
  source 1..19: mol_type = other DNA; note = ZmTAR4-1TAR4-1-asgRNA1
tacagatgggccggaacca

SEQ ID NO: 26 - DNA, 19 bp, synthetic construct
  source 1..19: mol_type = other DNA; note = sgRNA2
gagaagcgcgtatgcctgt

SEQ ID NO: 27 - DNA, 19 bp, synthetic construct
  source 1..19: mol_type = other DNA; note = ZmTAA1TAA1-asgRNA1
ttagcggtgaggaggcgcg

SEQ ID NO: 28 - DNA, 19 bp, synthetic construct
  source 1..19: mol_type = other DNA; note = sgRNA2
ggcccaccaaaaggccgcg

SEQ ID NO: 29 - DNA, 19 bp, synthetic construct
  source 1..19: mol_type = other DNA; note = Yucca2Yucca2-asgRNA1
tagcgcgccaacaccaccg

SEQ ID NO: 30 - DNA, 19 bp, synthetic construct
  source 1..19: mol_type = other DNA; note = sgRNA2
tcgagctcaaggagacaca

SEQ ID NO: 31 - DNA, 19 bp, synthetic construct
  source 1..19: mol_type = other DNA; note = Yucca4Yucca4-asgRNA1
ggagaagaggctatccatg

SEQ ID NO: 32 - DNA, 19 bp, synthetic construct
  source 1..19: mol_type = other DNA; note = sgRNA2
gacttgtactcgctcacgt

SEQ ID NO: 33 - DNA, 19 bp, synthetic construct
  source 1..19: mol_type = other DNA; note = ZmSIZ1aSIZ1a-asgRNA1
gtgttctatatgatccaag

SEQ ID NO: 34 - DNA, 19 bp, synthetic construct
  source 1..19: mol_type = other DNA; note = sgRNA2
atcagagaagaatagcgag

SEQ ID NO: 35 - DNA, 19 bp, synthetic construct
  source 1..19: mol_type = other DNA; note = SIZ1a-bsgRNA1
aacatatcagttaccacgt

SEQ ID NO: 36 - DNA, 19 bp, synthetic construct
  source 1..19: mol_type = other DNA; note = sgRNA2
tgtcttgatgccagctgat

SEQ ID NO: 37 - DNA, 19 bp, synthetic construct
  source 1..19: mol_type = other DNA; note = ZmSIZ1bSIZ1b-asgRNA1
tcttgatcgttactgtgga

SEQ ID NO: 38 - DNA, 19 bp, synthetic construct
  source 1..19: mol_type = other DNA; note = SIZ1b-bsgRNA1
cgcaacaagaacagatgaa

SEQ ID NO: 39 - DNA, 19 bp, synthetic construct
  source 1..19: mol_type = other DNA; note = sgRNA2
ccagcgcctttcaatcgcc

SEQ ID NO: 40 - DNA, 19 bp, synthetic construct
  source 1..19: mol_type = other DNA; note = ZmFER1FER1-asgRNA1
gtcgaggagaagaaaagaa

SEQ ID NO: 41 - DNA, 19 bp, synthetic construct
  source 1..19: mol_type = other DNA; note = sgRNA2
gaaacagcagctaaggcca

SEQ ID NO: 42 - DNA, 19 bp, synthetic construct
  source 1..19: mol_type = other DNA; note = ZmTSB2TSB2-asgRNA1
caggcgtggtacaacgccg

SEQ ID NO: 43 - DNA, 19 bp, synthetic construct
  source 1..19: mol_type = other DNA; note = sgRNA2
ggcacttcgattcgcaggt

SEQ ID NO: 44 - DNA, 19 bp, synthetic construct
  source 1..19: mol_type = other DNA; note = ZmTSB1TSB1-asgRNA1
ctcatctacctcaagaggg

SEQ ID NO: 45 - DNA, 19 bp, synthetic construct
  source 1..19: mol_type = other DNA; note = sgRNA2
gagatggggaacggcgccg

SEQ ID NO: 46 - DNA, 19 bp, synthetic construct
  source 1..19: mol_type = other DNA; note = ZmIGSIGS-asgRNA1
acgccttcacctcgcacag

SEQ ID NO: 47 - DNA, 19 bp, synthetic construct
  source 1..19: mol_type = other DNA; note = sgRNA2
ctcgcacctacgagaagaa

SEQ ID NO: 48 - DNA, 19 bp, synthetic construct
  source 1..19: mol_type = other DNA; note = ZmAUX2AUX2-asgRNA1
gcgagcaagggccattgcg

SEQ ID NO: 49 - DNA, 19 bp, synthetic construct
  source 1..19: mol_type = other DNA; note = sgRNA2
gcgacatcagcaaatggag

SEQ ID NO: 50 - DNA, 19 bp, synthetic construct
  source 1..19: mol_type = other DNA; note = ZmTAR2TAR2-asgRNA1
cagcactacgtcagtagca

SEQ ID NO: 51 - DNA, 19 bp, synthetic construct
  source 1..19: mol_type = other DNA; note = sgRNA2
caggcgcagagtcgtcggg

SEQ ID NO: 52 - DNA, 19 bp, synthetic construct
  source 1..19: mol_type = other DNA; note = ZmYUC6YUC6-asgRNA1
tacgcggcgaggttcctgg

SEQ ID NO: 53 - DNA, 19 bp, synthetic construct
  source 1..19: mol_type = other DNA; note = sgRNA2
cacgcagtggagtaccggt

SEQ ID NO: 54 - DNA, 27 bp, synthetic construct
  source 1..27: mol_type = other DNA; note = SG1F
attgggccccctgatctcgcgcggttt

SEQ ID NO: 55 - DNA, 27 bp, synthetic construct
  source 1..27: mol_type = other DNA; note = SG1R
tctaaaaccgcgcgagatcagggggcc

SEQ ID NO: 56 - DNA, 28 bp, synthetic construct
  source 1..28: mol_type = other DNA; note = SG2F
accgaattggcaatataaaattaccgat

SEQ ID NO: 57 - DNA, 28 bp, synthetic construct
  source 1..28: mol_type = other DNA; note = SG2R
aaacatcggtaattttatattgccaatt

SEQ ID NO: 58 - DNA, 27 bp, synthetic construct
  source 1..27: mol_type = other DNA; note = SG1F
attgcaccacgcgcgagatcagggttt

SEQ ID NO: 59 - DNA, 27 bp, synthetic construct
  source 1..27: mol_type = other DNA; note = SG1R
tctaaaacggccccctgatctcgcgcg

SEQ ID NO: 60 - DNA, 28 bp, synthetic construct
  source 1..28: mol_type = other DNA; note = SG2F
accgaattgataagaacaccgaacagca

SEQ ID NO: 61 - DNA, 28 bp, synthetic construct
  source 1..28: mol_type = other DNA; note = SG2R
aaactgctgttcggtgttcttatcaatt

SEQ ID NO: 62 - DNA, 27 bp, synthetic construct
  source 1..27: mol_type = other DNA; note = SG1F
attgacaaaacaagaacctacaagttt

SEQ ID NO: 63 - DNA, 27 bp, synthetic construct
  source 1..27: mol_type = other DNA; note = SG1R
tctaaaacttgtaggttcttgttttgt

SEQ ID NO: 64 - DNA, 28 bp, synthetic construct
  source 1..28: mol_type = other DNA; note = SG2F
accgaattgtacatactagcagcacaaa

SEQ ID NO: 65 - DNA, 28 bp, synthetic construct
  source 1..28: mol_type = other DNA; note = SG2R
aaactttgtgctgctagtatgtacaatt

SEQ ID NO: 66 - DNA, 27 bp, synthetic construct
  source 1..27: mol_type = other DNA; note = SG1F
attgggatcgtgtaaccttggtagttt

SEQ ID NO: 67 - DNA, 27 bp, synthetic construct
  source 1..27: mol_type = other DNA; note = SG1R
tctaaaacggatcgtgtaaccttggta

SEQ ID NO: 68 - DNA, 28 bp, synthetic construct
  source 1..28: mol_type = other DNA; note = SG2F
accgaattggttgatccaaagcaggtaa

SEQ ID NO: 69 - DNA, 28 bp, synthetic construct
  source 1..28: mol_type = other DNA; note = SG2R
aaacttacctgctttggatcaaccaatt

SEQ ID NO: 70 - DNA, 27 bp, synthetic construct
  source 1..27: mol_type = other DNA; note = SG1F
attgtttcaaagcagcaaccacagttt

SEQ ID NO: 71 - DNA, 27 bp, synthetic construct
  source 1..27: mol_type = other DNA; note = SG1R
tctaaaactgtggttgctgctttgaaa

SEQ ID NO: 72 - DNA, 28 bp, synthetic construct
  source 1..28: mol_type = other DNA; note = SG2F
accgaattgtacatgtagagggagagat

SEQ ID NO: 73 - DNA, 28 bp, synthetic construct
  source 1..28: mol_type = other DNA; note = SG2R
aaacatctctccctctacatgtacaatt

SEQ ID NO: 74 - DNA, 27 bp, synthetic construct
  source 1..27: mol_type = other DNA; note = SG1F
attgagcacctgcaacacgccaagttt

SEQ ID NO: 75 - DNA, 27 bp, synthetic construct
  source 1..27: mol_type = other DNA; note = SG1R
tctaaaacttggcgtgttgcaggtgct

SEQ ID NO: 76 - DNA, 28 bp, synthetic construct
  source 1..28: mol_type = other DNA; note = SG2F
accgaattgacttggcacatgagatgca

SEQ ID NO: 77 - DNA, 28 bp, synthetic construct
  source 1..28: mol_type = other DNA; note = SG2R
aaactgcatctcatgtgccaagtcaatt

SEQ ID NO: 78 - DNA, 27 bp, synthetic construct
  source 1..27: mol_type = other DNA; note = SG1F
attgtgtgtccttcaatgttgtagttt

SEQ ID NO: 79 - DNA, 27 bp, synthetic construct
  source 1..27: mol_type = other DNA; note = SG1R
tctaaaactacaacattgaaggacaca

SEQ ID NO: 80 - DNA, 28 bp, synthetic construct
  source 1..28: mol_type = other DNA; note = SG2F
accgaattgtctaacacataggatgcgg

SEQ ID NO: 81 - DNA, 28 bp, synthetic construct
  source 1..28: mol_type = other DNA; note = SG2R
aaacccgcatcctatgtgttagacaatt

SEQ ID NO: 82 - DNA, 27 bp, synthetic construct
  source 1..27: mol_type = other DNA; note = SG1F
attgtattcgtcaggcacaaaaggttt

SEQ ID NO: 83 - DNA, 27 bp, synthetic construct
  source 1..27: mol_type = other DNA; note = SG1R
tctaaaaccttttgtgcctgacgaata

SEQ ID NO: 84 - DNA, 28 bp, synthetic construct
  source 1..28: mol_type = other DNA; note = SG2F
accgaattgcaatccactccattctaca

SEQ ID NO: 85 - DNA, 28 bp, synthetic construct
  source 1..28: mol_type = other DNA; note = SG2R
aaactgtagaatggagtggattgcaatt

SEQ ID NO: 86 - DNA, 27 bp, synthetic construct
  source 1..27: mol_type = other DNA; note = SG1F
attgcaccgaccagatgatgcgagttt

SEQ ID NO: 87 - DNA, 27 bp, synthetic construct
  source 1..27: mol_type = other DNA; note = SG1R
tctaaaactcgcatcatctggtcggtg

SEQ ID NO: 88 - DNA, 28 bp, synthetic construct
  source 1..28: mol_type = other DNA; note = SG2F
accgaattgttaacaaagaaataaactg

SEQ ID NO: 89 - DNA, 28 bp, synthetic construct
  source 1..28: mol_type = other DNA; note = SG2R
aaaccagtttatttctttgttaacaatt

SEQ ID NO: 90 - DNA, 27 bp, synthetic construct
  source 1..27: mol_type = other DNA; note = SG1F
attgctccctcgcatcatctggtgttt

SEQ ID NO: 91 - DNA, 27 bp, synthetic construct
  source 1..27: mol_type = other DNA; note = SG1R
tctaaaacaccagatgatgcgagggag

SEQ ID NO: 92 - DNA, 28 bp, synthetic construct
  source 1..28: mol_type = other DNA; note = SG2F
accgaattgcatcaagcagatataacgc

SEQ ID NO: 93 - DNA, 28 bp, synthetic construct
  source 1..28: mol_type = other DNA; note = SG2R
aaacgcgttatatctgcttgatgcaatt

SEQ ID NO: 94 - DNA, 27 bp, synthetic construct
  source 1..27: mol_type = other DNA; note = SG1F
attgcggagaacctgaatccgaagttt

SEQ ID NO: 95 - DNA, 27 bp, synthetic construct
  source 1..27: mol_type = other DNA; note = SG1R
tctaaaacttcggattcaggttctccg

SEQ ID NO: 96 - DNA, 28 bp, synthetic construct
  source 1..28: mol_type = other DNA; note = SG2F
accgaattgtatcttgacagtcacggtg

SEQ ID NO: 97 - DNA, 28 bp, synthetic construct
  source 1..28: mol_type = other DNA; note = SG2R
aaaccaccgtgactgtcaagatacaatt

SEQ ID NO: 98 - DNA, 27 bp, synthetic construct
  source 1..27: mol_type = other DNA; note = SG1F
attgatcacctgaacatggtgcggttt

SEQ ID NO: 99 - DNA, 27 bp, synthetic construct
  source 1..27: mol_type = other DNA; note = SG1R
tctaaaaccgcaccatgttcaggtgat

SEQ ID NO: 100 - DNA, 28 bp, synthetic construct
  source 1..28: mol_type = other DNA; note = SG2F
accgaattgcagagccaccctcgccggt

SEQ ID NO: 101 - DNA, 28 bp, synthetic construct
  source 1..28: mol_type = other DNA; note = SG2R
aaacaccggcgagggtggctctgcaatt

SEQ ID NO: 102 - DNA, 27 bp, synthetic construct
  source 1..27: mol_type = other DNA; note = SG1F
attgtacagatgggccggaaccagttt

SEQ ID NO: 103 - DNA, 27 bp, synthetic construct
  source 1..27: mol_type = other DNA; note = SG1R
tctaaaactggttccggcccatctgta

SEQ ID NO: 104 - DNA, 28 bp, synthetic construct
  source 1..28: mol_type = other DNA; note = SG2F
accgaattggagaagcgcgtatgcctgt

SEQ ID NO: 105 - DNA, 28 bp, synthetic construct
  source 1..28: mol_type = other DNA; note = SG2R
aaacacaggcatacgcgcttctccaatt

SEQ ID NO: 106 - DNA, 27 bp, synthetic construct
  source 1..27: mol_type = other DNA; note = SG1F
attgttagcggtgaggaggcgcggttt

SEQ ID NO: 107 - DNA, 27 bp, synthetic construct
  source 1..27: mol_type = other DNA; note = SG1R
tctaaaaccgcgcctcctcaccgctaa

SEQ ID NO: 108 - DNA, 28 bp, synthetic construct
  source 1..28: mol_type = other DNA; note = SG2F
accgaattgggcccaccaaaaggccgcg

SEQ ID NO: 109 - DNA, 28 bp, synthetic construct
  source 1..28: mol_type = other DNA; note = SG2R
aaaccgcggccttttggtgggcccaatt

SEQ ID NO: 110 - DNA, 27 bp, synthetic construct
  source 1..27: mol_type = other DNA; note = SG1F
attgtagcgcgccaacaccaccggttt

SEQ ID NO: 111 - DNA, 27 bp, synthetic construct
  source 1..27: mol_type = other DNA; note = SG1R
tctaaaaccggtggtgttggcgcgcta

SEQ ID NO: 112 - DNA, 28 bp, synthetic construct
  source 1..28: mol_type = other DNA; note = SG2F
accgaattgtcgagctcaaggagacaca

SEQ ID NO: 113 - DNA, 28 bp, synthetic construct
  source 1..28: mol_type = other DNA; note = SG2R
aaactgtgtctccttgagctcgacaatt

SEQ ID NO: 114 - DNA, 27 bp, synthetic construct
  source 1..27: mol_type = other DNA; note = SG1F
attgggagaagaggctatccatggttt

SEQ ID NO: 115 - DNA, 27 bp, synthetic construct
  source 1..27: mol_type = other DNA; note = SG1R
tctaaaaccatggatagcctcttctcc

SEQ ID NO: 116 - DNA, 28 bp, synthetic construct
  source 1..28: mol_type = other DNA; note = SG2F
accgaattggacttgtactcgctcacgt

SEQ ID NO: 117 - DNA, 28 bp, synthetic construct
  source 1..28: mol_type = other DNA; note = SG2R
aaacacgtgagcgagtacaagtccaatt

SEQ ID NO: 118 - DNA, 27 bp, synthetic construct
  source 1..27: mol_type = other DNA; note = SG1F
attggtgttctatatgatccaaggttt

SEQ ID NO: 119 - DNA, 27 bp, synthetic construct
  source 1..27: mol_type = other DNA; note = SG1R
tctaaaaccttggatcatatagaacac

SEQ ID NO: 120 - DNA, 28 bp, synthetic construct
  source 1..28: mol_type = other DNA; note = SG2F
accgaattgatcagagaagaatagcgag

SEQ ID NO: 121 - DNA, 29 bp, synthetic construct
  source 1..29: mol_type = other DNA; note = SG2R
aaacactcgctattcttctctgatcaatt

SEQ ID NO: 122 - DNA, 27 bp, synthetic construct
  source 1..27: mol_type = other DNA; note = SG1F
attgaacatatcagttaccacgtgttt

SEQ ID NO: 123 - DNA, 27 bp, synthetic construct
  source 1..27: mol_type = other DNA; note = SG1R
tctaaaacacgtggtaactgatatgtt

SEQ ID NO: 124 - DNA, 28 bp, synthetic construct
  source 1..28: mol_type = other DNA; note = SG2F
accgaattgtgtcttgatgccagctgat

SEQ ID NO: 125 - DNA, 28 bp, synthetic construct
  source 1..28: mol_type = other DNA; note = SG2R
aaacatcagctggcatcaagacacaatt

SEQ ID NO: 126 - DNA, 27 bp, synthetic construct
  source 1..27: mol_type = other DNA; note = SG1F
attgtcttgatcgttactgtggagttt

SEQ ID NO: 127 - DNA, 27 bp, synthetic construct
  source 1..27: mol_type = other DNA; note = SG1R
tctaaaactccacagtaacgatcaaga

SEQ ID NO: 128 - DNA, 28 bp, synthetic construct
  source 1..28: mol_type = other DNA; note = SG2F
accgaattggtgttctatatgatccaag

SEQ ID NO: 129 - DNA, 28 bp, synthetic construct
  source 1..28: mol_type = other DNA; note = SG2R
aaaccttggatcatatagaacaccaatt

SEQ ID NO: 130 - DNA, 27 bp, synthetic construct
  source 1..27: mol_type = other DNA; note = SG1F
attgcgcaacaagaacagatgaagttt

SEQ ID NO: 131 - DNA, 27 bp, synthetic construct
  source 1..27: mol_type = other DNA; note = SG1R
tctaaaacttcatctgttcttgttgcg

SEQ ID NO: 132 - DNA, 28 bp, synthetic construct
  source 1..28: mol_type = other DNA; note = SG2F
accgaattgccagcgcctttcaatcgcc

SEQ ID NO: 133 - DNA, 28 bp, synthetic construct
  source 1..28: mol_type = other DNA; note = SG2R
aaacggcgattgaaaggcgctggcaatt

SEQ ID NO: 134 - DNA, 27 bp, synthetic construct
  source 1..27: mol_type = other DNA; note = SG1F
attggtcgaggagaagaaaagaagttt

SEQ ID NO: 135 - DNA, 27 bp, synthetic construct
  source 1..27: mol_type = other DNA; note = SG1R
tctaaaacttcttttcttctcctcgac

SEQ ID NO: 136 - DNA, 28 bp, synthetic construct
  source 1..28: mol_type = other DNA; note = SG2F
accgaattggaaacagcagctaaggcca

SEQ ID NO: 137 - DNA, 28 bp, synthetic construct
  source 1..28: mol_type = other DNA; note = SG2R
aaactggccttagctgctgtttccaatt

SEQ ID NO: 138 - DNA, 27 bp, synthetic construct
  source 1..27: mol_type = other DNA; note = SG1F
attgcaggcgtggtacaacgccggttt

SEQ ID NO: 139 - DNA, 27 bp, synthetic construct
  source 1..27: mol_type = other DNA; note = SG1R
tctaaaaccggcgttgtaccacgcctg

SEQ ID NO: 140 - DNA, 28 bp, synthetic construct
  source 1..28: mol_type = other DNA; note = SG2F
accgaattgggcacttcgattcgcaggt

SEQ ID NO: 141 - DNA, 28 bp, synthetic construct
  source 1..28: mol_type = other DNA; note = SG2R
aaacacctgcgaatcgaagtgcccaatt

SEQ ID NO: 142 - DNA, 27 bp, synthetic construct
  source 1..27: mol_type = other DNA; note = SG1F
attgctcatctacctcaagaggggttt

SEQ ID NO: 143 - DNA, 27 bp, synthetic construct
  source 1..27: mol_type = other DNA; note = SG1R
tctaaaacccctcttgaggtagatgag

SEQ ID NO: 144 - DNA, 28 bp, synthetic construct
  source 1..28: mol_type = other DNA; note = SG2F
accgaattggagatggggaacggcgccg

SEQ ID NO: 145 - DNA, 28 bp, synthetic construct
  source 1..28: mol_type = other DNA; note = SG2R
aaaccggcgccgttccccatctccaatt

SEQ ID NO: 146 - DNA, 27 bp, synthetic construct
  source 1..27: mol_type = other DNA; note = SG1F
attgacgccttcacctcgcacaggttt

SEQ ID NO: 147 - DNA, 27 bp, synthetic construct
  source 1..27: mol_type = other DNA; note = SG1R
tctaaaacctgtgcgaggtgaaggcgt

SEQ ID NO: 148 - DNA, 28 bp, synthetic construct
  source 1..28: mol_type = other DNA; note = SG2F
accgaattgctcgcacctacgagaagaa

SEQ ID NO: 149 - DNA, 28 bp, synthetic construct
  source 1..28: mol_type = other DNA; note = SG2R
aaacttcttctcgtaggtgcgagcaatt

SEQ ID NO: 150 - DNA, 27 bp, synthetic construct
  source 1..27: mol_type = other DNA; note = SG1F
attggcgagcaagggccattgcggttt

SEQ ID NO: 151 - DNA, 27 bp, synthetic construct
  source 1..27: mol_type = other DNA; note = SG1R
tctaaaaccgcaatggcccttgctcgc

SEQ ID NO: 152 - DNA, 28 bp, synthetic construct
  source 1..28: mol_type = other DNA; note = SG2F
accgaattggcgacatcagcaaatggag

SEQ ID NO: 153 - DNA, 28 bp, synthetic construct
  source 1..28: mol_type = other DNA; note = SG2R
aaacctccatttgctgatgtcgccaatt

SEQ ID NO: 154 - DNA, 27 bp, synthetic construct
  source 1..27: mol_type = other DNA; note = SG1F
attgcagcactacgtcagtagcagttt

SEQ ID NO: 155 - DNA, 27 bp, synthetic construct
  source 1..27: mol_type = other DNA; note = SG1R
tctaaaactgctactgacgtagtgctg

SEQ ID NO: 156 - DNA, 28 bp, synthetic construct
  source 1..28: mol_type = other DNA; note = SG2F
accgaattgcaggcgcagagtcgtcggg

SEQ ID NO: 157 - DNA, 28 bp, synthetic construct
  source 1..28: mol_type = other DNA; note = SG2R
aaaccccgacgactctgcgcctgcaatt

SEQ ID NO: 158 - DNA, 27 bp, synthetic construct
  source 1..27: mol_type = other DNA; note = SG1F
attgtacgcggcgaggttcctgggttt

SEQ ID NO: 159 - DNA, 27 bp, synthetic construct
  source 1..27: mol_type = other DNA; note = SG1R
tctaaaacccaggaacctcgccgcgta

SEQ ID NO: 160 - DNA, 28 bp, synthetic construct
  source 1..28: mol_type = other DNA; note = SG2F
accgaattgcacgcagtggagtaccggt

SEQ ID NO: 161 - DNA, 28 bp, synthetic construct
  source 1..28: mol_type = other DNA; note = SG2R
aaacaccggtactccactgcgtgcaatt

SEQ ID NO: 162 - DNA, 19 bp, synthetic construct
  source 1..19: mol_type = other DNA; note = RZmGBSSIZmGBSSI-F
cagtgcaacgtcgtggagc

SEQ ID NO: 163 - DNA, 22 bp, synthetic construct
  source 1..22: mol_type = other DNA; note = ZmGBSSI-R
cgtaggattcagaaagaaaatg

SEQ ID NO: 164 - DNA, 20 bp, synthetic construct
  source 1..20: mol_type = other DNA; note = ZmSH2ZmSH2-F
gtgacagggcatccaagagg

SEQ ID NO: 165 - DNA, 22 bp, synthetic construct
  source 1..22: mol_type = other DNA; note = ZmSH2-R
catcgaagccagatcccaaaag

SEQ ID NO: 166 - DNA, 21 bp, synthetic construct
  source 1..21: mol_type = other DNA; note = ZmAGPLS3ZmAGPLS3-F
cctgctacctacagttcctgg

SEQ ID NO: 167 - DNA, 19 bp, synthetic construct
  source 1..19: mol_type = other DNA; note = ZmAGPLS3-R
gcatcaataggcaattagg

SEQ ID NO: 168 - DNA, 19 bp, synthetic construct
  source 1..19: mol_type = other DNA; note = Bt2Bt2-F
ctgttggagatcatcggag

SEQ ID NO: 169 - DNA, 19 bp, synthetic construct
  source 1..19: mol_type = other DNA; note = Bt2-R
ccctaaccctaatcctctg

SEQ ID NO: 170 - DNA, 19 bp, synthetic construct
  source 1..19: mol_type = other DNA; note = ZmDOF36ZmDOF36-F
cagtgccagccacaggagg

SEQ ID NO: 171 - DNA, 19 bp, synthetic construct
  source 1..19: mol_type = other DNA; note = ZmDOF36-R
gcacagagtttgtaaatgc

SEQ ID NO: 172 - DNA, 21 bp, synthetic construct
  source 1..21: mol_type = other DNA; note = ZmTPS9ZmTPS9-F
gtcatcgtctcccaccgtctg

SEQ ID NO: 173 - DNA, 19 bp, synthetic construct
  source 1..19: mol_type = other DNA; note = ZmTPS9-R
ccatcgccaggaacttgag

SEQ ID NO: 174 - DNA, 22 bp, synthetic construct
  source 1..22: mol_type = other DNA; note = ZmAuxRP1ZmAuxRP1-F
gacgatggcttattttctgctc

SEQ ID NO: 175 - DNA, 21 bp, synthetic construct
  source 1..21: mol_type = other DNA; note = ZmAuxRP1-R
ctgtccgtcatcacgttgtcc

SEQ ID NO: 176 - DNA, 19 bp, synthetic construct
  source 1..19: mol_type = other DNA; note = ZmTAR4-1ZmTAR4-1-F
ggtgcgagtgcaacagctg

SEQ ID NO: 177 - DNA, 21 bp, synthetic construct
  source 1..21: mol_type = other DNA; note = ZmTAR4-1-R
gatggcttggagacggtgaac

SEQ ID NO: 178 - DNA, 20 bp, synthetic construct
  source 1..20: mol_type = other DNA; note = ZmTAA1ZmTAA1-F
cacgagcaggctgctgtagg

SEQ ID NO: 179 - DNA, 22 bp, synthetic construct
  source 1..22: mol_type = other DNA; note = ZmTAA1-R
gtgcgacggagctgtgctgttg

SEQ ID NO: 180 - DNA, 20 bp, synthetic construct
  source 1..20: mol_type = other DNA; note = Yucca2Yucca2-F
cgcatggacagcctcttctc

SEQ ID NO: 181 - DNA, 21 bp, synthetic construct
  source 1..21: mol_type = other DNA; note = Yucca2-R
cagggacgacggcgatatctc

SEQ ID NO: 182 - DNA, 22 bp, synthetic construct
  source 1..22: mol_type = other DNA; note = Yucca4Yucca4-F
caccttctcctcctcattcttc

SEQ ID NO: 183 - DNA, 22 bp, synthetic construct
  source 1..22: mol_type = other DNA; note = Yucca4-R
caggtccagcgacacctccatc

SEQ ID NO: 184 - DNA, 22 bp, synthetic construct
  source 1..22: mol_type = other DNA; note = ZmSIZ1a-F1
gattcagagtagttcggacatc

SEQ ID NO: 185 - DNA, 20 bp, synthetic construct
  source 1..20: mol_type = other DNA; note = ZmSIZ1a-R1
cagccacctcctatctcatc

SEQ ID NO: 186 - DNA, 22 bp, synthetic construct
  source 1..22: mol_type = other DNA; note = ZmSIZ1a-F2
cgatgtgagtgcctgtagtttc

SEQ ID NO: 187 - DNA, 22 bp, synthetic construct
  source 1..22: mol_type = other DNA; note = ZmSIZ1a-R2
gcaacctgcattctactgttag

SEQ ID NO: 188 - DNA, 20 bp, synthetic construct
  source 1..20: mol_type = other DNA; note = ZmSIZ1b-F1
cactgggatagctgaatttg

SEQ ID NO: 189 - DNA, 19 bp, synthetic construct
  source 1..19: mol_type = other DNA; note = ZmSIZ1b-R1
catctccaatgccgtgttc

SEQ ID NO: 190 - DNA, 19 bp, synthetic construct
  source 1..19: mol_type = other DNA; note = ZmSIZ1b-F2
gtatttagggtttgggtgc

SEQ ID NO: 191 - DNA, 22 bp, synthetic construct
  source 1..22: mol_type = other DNA; note = ZmSIZ1b-R2
ctttaacaacctgtcttctacc

SEQ ID NO: 192 - DNA, 23 bp, synthetic construct
  source 1..23: mol_type = other DNA; note = ZmFER1ZmFER1-F
cttgcatcatttcatgtccctac

SEQ ID NO: 193 - DNA, 19 bp, synthetic construct
  source 1..19: mol_type = other DNA; note = ZmFER1-R
caacatcgctgccatagtc

SEQ ID NO: 194 - DNA, 19 bp, synthetic construct
  source 1..19: mol_type = other DNA; note = ZmTSB2ZmTSB2-F
ggtcgcacaagcccaacac

SEQ ID NO: 195 - DNA, 21 bp, synthetic construct
  source 1..21: mol_type = other DNA; note = ZmTSB2-R
cgagatcagaggtgccattcc

SEQ ID NO: 196 - DNA, 19 bp, synthetic construct
  source 1..19: mol_type = other DNA; note = ZmTSB1-F1
cctcccactcacgctttcc

SEQ ID NO: 197 - DNA, 21 bp, synthetic construct
  source 1..21: mol_type = other DNA; note = ZmTSB1-R1
cacagcattgttgatcttgtg

SEQ ID NO: 198 - DNA, 19 bp, synthetic construct
  source 1..19: mol_type = other DNA; note = ZmTSB1-F2
cccagccagcctatctacc

SEQ ID NO: 199 - DNA, 19 bp, synthetic construct
  source 1..19: mol_type = other DNA; note = ZmTSB1-R2
ctacctggaactcgtcgtc

SEQ ID NO: 200 - DNA, 20 bp, synthetic construct
  source 1..20: mol_type = other DNA; note = ZmIGSZmIGS-F
gaggtgatccagtgggagag

SEQ ID NO: 201 - DNA, 19 bp, synthetic construct
  source 1..19: mol_type = other DNA; note = ZmIGS-R
ggtcaggatgctcaggcac

SEQ ID NO: 202 - DNA, 21 bp, synthetic construct
  source 1..21: mol_type = other DNA; note = ZmAUX2ZmAUX2-F
gcagcatcacaaagcaaactg

SEQ ID NO: 203 - DNA, 21 bp, synthetic construct
  source 1..21: mol_type = other DNA; note = ZmAUX2-R
ccaaactcctcctctgacatc

SEQ ID NO: 204 - DNA, 20 bp, synthetic construct
  source 1..20: mol_type = other DNA; note = ZmTAR2ZmTAR2-F
gcattacaccttggcacgtc

SEQ ID NO: 205 - DNA, 19 bp, synthetic construct
  source 1..19: mol_type = other DNA; note = ZmTAR2-R
gcacggacagcggatcctc

SEQ ID NO: 206 - DNA, 19 bp, synthetic construct
  source 1..19: mol_type = other DNA; note = ZmYUC6ZmYUC6-F
gtacctgcaccgcgacgac

SEQ ID NO: 207 - DNA, 20 bp, synthetic construct
  source 1..20: mol_type = other DNA; note = ZmYUC6-R
gtaccagatcgagcagcagg

(19) United States
(12) Patent Application Publication
(10) Pub. No.: US 2026/0226487 A1
(43) Pub. Date: Aug. 6, 2026

(54) METHOD FOR PRODUCING LOW-MYCOTOXIN, HIGH-STARCH MAIZE GERMPLASM USING GENE EDITING, AND APPLICATIONS THEREOF

(71) Applicants: SHANDONG ACADEMY OF AGRICULTURAL SCIENCES, Jinan (CN); SHANDONG DENGHAI SEEDS CO., LTD., Laizhou (CN); HUAZHONG AGRICULTURAL UNIVERSITY, Wuhan (CN); CHANGZHOU XINMI BIOTECHNOLOGY CO., LTD., Changzhou (CN)

(72) Inventors: Maolin ZHANG, Jinan (CN); Zhaohua DING, Jinan (CN); Jianbing YAN, Wuhan (CN); Shiwei TANG, Laizhou (CN); Tieshan LIU, Jinan (CN); Liming WANG, Jinan (CN); Chunmei HE, Jinan (CN); Juan WANG, Jinan (CN); Haiying GUAN, Jinan (CN); Chunxiao LIU, Jinan (CN); Rui DONG, Jinan (CN); Qiang LIU, Jinan (CN); Jieting XU, Changzhou (CN); Youbo WEI, Changzhou (CN); Runqing YUE, Jinan (CN)

(21) Appl. No.: 19/434,459

(22) Filed: Dec. 29, 2025

(30) Foreign Application Priority Data
Dec. 31, 2024 (CN) 202411992585X

(51) Int. Cl. C12N 15/82; A01H 4/00; C12N 9/22; C12N 15/11

(52) U.S. Cl. CPC C12N 15/8213; A01H 4/008; C12N 9/226; C12N 15/11; C12N 15/8205; C12N 15/8274; C12N 2310/20

(57) ABSTRACT

A method for producing maize germplasm with low mycotoxin content and high starch content using gene editing technology, and applications thereof. The method comprises using CRISPR/Cas9 multi-gene editing technology and genetic transformation, wherein, during genetic transformation, an infection medium contains one or more of betaine and calcium chloride. Based on existing studies of functional genes related to starch synthesis and ear rot, the present invention applications high-throughput gene editing to efficiently and randomly aggregate numerous advantageous loci. By adding appropriate concentrations of betaine and calcium chloride during genetic transformation, a higher number of loci are edited in a single transgenic event with better efficiency, thereby effectively increasing diversity and producing more superior locus combination patterns. Numerous novel variant germplasm having high starch content and resistance to ear rot and other diseases are generated efficiently and at scale, and the method has good application value in gene editing-assisted breeding.

### SEQUENCE LISTING

The contents of the electronic sequence listing (file name: “PCPIP202536US_sequence_listing_ST26.xml”; size: 219,450 bytes; date of production: <Dec. 19, 2025>), submitted herewith in XML format in accordance with WIPO Standard ST.26, are incorporated by reference in their entirety.

### CROSS-REFERENCE TO RELATED APPLICATIONS

The present invention claims priority to Chinese Patent Application No. CN202411992585.X, filed on Dec. 31, 2024, entitled “METHOD FOR PRODUCING LOW-MYCOTOXIN, HIGH-STARCH MAIZE GERMPLASM USING GENE EDITING, AND APPLICATIONS THEREOF”. The entire contents of the above-identified application are incorporated herein by reference and made a part of this application for all purposes.

### TECHNICAL FIELD

The present invention relates to the field of genetic breeding technologies, and in particular to a method for producing maize germplasm with low mycotoxin content and high starch content using gene editing technology, and applications thereof.

### BACKGROUND OF THE INVENTION

Any discussion of the prior art throughout the specification should not be taken as an admission that such prior art is widely known or forms part of the common general knowledge in the art.

Gene editing can introduce point mutations in a gene coding region to weaken or eliminate gene function. In addition, a dual-target system can be used to delete fragments in promoter elements and untranslated regions (UTRs), thereby regulating gene expression, and in turn, regulating gene function. In recent years, the CRISPR/Cas9 system has been widely used for gene editing in a variety of species due to its high targeting specificity and its simplicity and convenience in operation, and large-scale mutant resources have been generated in rice and soybean. However, these large-scale gene-editing studies mainly treat CRISPR/Cas9 as an alternative to conventional mutagenesis. There remains a lack of in-depth research on how to establish a high-throughput workflow tailored to plant characteristics from target design to mutant sequence detection, to reduce cost, and to explore the patterns of genome impacts after applying this emerging technology. Professor Yan Jianbing at Huazhong Agricultural University used high-throughput single-target gene editing technology to perform high-throughput gene editing on the coding regions of hundreds of genes, thereby obtaining mutant plants with various phenotypes, including agronomic traits such as plant height, ear position, stress resistance, and kernel traits, which can enable validation of multiple gene functions. However, the gene-editing efficiency still needs to be further improved. Moreover, the technology has been mostly used for gene function validation and has not yet achieved high-throughput creation of materials. Only by achieving high-throughput creation of superior materials can the required breeding materials be selected therefrom, thereby truly realizing genetic improvement of maize varieties.

At present, high-throughput gene editing is mostly used for gene function validation and has not yet achieved high-throughput creation of breeding materials. Only by achieving high-throughput creation of superior materials can optimal breeding materials be selected therefrom, thereby truly realizing genetic improvement of maize varieties. As maize genetic transformation efficiency continues to be optimized and improved, the number of functional genes validated continues to increase, and it becomes possible to improve traits in a targeted manner through gene editing technology. However, current gene editing is mostly applied to functional modification of a single gene or a few genes, and cannot achieve large-scale and efficient aggregation of superior functions, resulting in a low innovation rate of novel germplasm and low selectivity, which limits the development of germplasm innovation.

Accordingly, there is a need for a method for efficiently producing novel maize germplasm using gene editing technology, so as to realize efficient, scalable aggregation of superior functional loci in maize and to improve both the innovation rate and selectivity of novel germplasm.

### SUMMARY OF THE INVENTION

To solve the above problems, the present invention provides a method for efficiently producing maize germplasm with low mycotoxin content and high starch content using gene editing technology, and applications thereof. During genetic transformation, by adding appropriate amounts of betaine and calcium chloride to an infection medium, the present invention greatly increases the probability of simultaneous multi-locus editing in a single transgenic event, while enabling diversified and efficient aggregation of advantageous loci for high starch content and low mycotoxin content (e.g., resistance to ear rot). This substantially improves the speed and efficiency for generating superior novel germplasm and represents a fast and efficient approach for generating new breeding materials. Based on the above research, the present patent has been completed.

To achieve the above objective, the present invention adopts the following technical solutions.

In a first aspect, the present invention provides a method for improving gene editing efficiency in maize. The method comprises using CRISPR/Cas9 multi-gene editing technology and genetic transformation, wherein, during the genetic transformation, an infection medium contains one or more of betaine and calcium chloride.

In some embodiments, the method comprises the following steps:
S1: selecting target genes and designing guide RNAs (gRNAs);
S2: using the CRISPR/Cas9 system to obtain dual-target knockout vectors by restriction enzyme digestion-ligation and genetic recombination, individually transforming the vectors into Agrobacterium, and mixing the Agrobacterium cultures in equal volumes to prepare a pooled Agrobacterium culture;
S3: genetic transformation: transforming a maize recipient material (e.g., maize embryos, particularly immature maize embryos) with the pooled Agrobacterium obtained in S2, and obtaining positive seedlings by herbicide-resistance selection, wherein the infection medium used during genetic transformation contains 80-120 mM betaine and/or 30-60 mM calcium chloride; and
S4: planting and selecting homozygous edited plants, calculating gene editing efficiency, and observing phenotypes.

In some embodiments, in step S1, the gRNAs are single guide RNAs (sgRNAs), based on maize genome sequence information, specific sgRNA sequences are designed for genes related to ear rot, low mycotoxin and auxin content (predicted to be related to low mycotoxin).

In step S2, the sgRNAs prepared in step S1 are integrated into a CRISPR/Cas9 vector, and after sequencing verification of correct construction, a plurality of dual-target knockout vectors are obtained.

The plurality of dual-target knockout vectors are individually transformed into Agrobacterium EHA105, and the Agrobacterium cultures are mixed in equal volumes to obtain a pooled culture for subsequent genetic transformation.

In some embodiments, the volume of the Agrobacterium EHA105 culture is 80-100 μL.

In some embodiments, in step S2, the CRISPR/Cas9 vector is a binary expression vector; preferably, the binary expression vector is pCXB053.

In some embodiments, the maize embryos are immature maize embryos, and wherein in step S3, the genetic transformation comprises the following steps:
S3-1: placing immature maize embryos into 1.5-2 ml of a suspension and treating for 25-35 min;
S3-2: removing the treatment solution, adding an infection medium, and allowing infection to proceed statically for 3-8 min;
S3-3: transferring infected immature maize embryos to a co-cultivation medium and culturing in the dark at 22-24° C. for 3 days, and then transferring the immature maize embryos to a resting medium and culturing in the dark at 27-29° C. for 5-7 days;
S3-4: after completion of culture in S3-3, removing embryonic roots from the immature maize embryos and transferring the embryos to a first selection medium containing 5 mg/L bialaphos for 14 days, and then transferring the immature maize embryos to a second selection medium containing 8 mg/L bialaphos for 14 days to obtain resistant callus; and
S3-5: transferring the resistant callus to a differentiation medium, culturing at 24-26° C. under light at 5,000 lux for 3 weeks, and obtaining regenerated seedlings.

In some embodiments, the infection medium may comprise ½ Murashige and Skoog (MS), sucrose 68.5 g/L, glucose 36 g/L, L-proline 0.115 g/L, acetosyringone 200 mM, cysteine 200 mg/L, a mixed Agrobacterium suspension having an OD600 of 0.5-1, and betaine 100 mM and/or calcium chloride 50 mM.

In some embodiments, the infection medium may comprise ½ MS, sucrose 68.5 g/L, glucose 36 g/L, L-proline 0.115 g/L, acetosyringone 200 mM, cysteine 200 mg/L, a mixed Agrobacterium suspension having an OD600 of 0.5-1, betaine 100 mM, and calcium chloride 50 mM.

In a second aspect, the present invention provides application of the above method in producing novel plant breeding germplasm.

In a third aspect, the present invention provides application of the above method in producing novel maize germplasm with low mycotoxin content and high starch content.

In a fourth aspect, the present invention provides a method for producing novel maize germplasm with low mycotoxin content and high starch content. The method comprises editing, using the above method, maize genes ZmGBSSI, Shrunken 2, Brittle 2, ZmDOF36, ZmTPS9, ZmAuxRP1, TAR4-1, TAR4-3, Yucca2, Yucca4, ZmSIZ1a, ZmSIZ1b, ZmFER1, ZmTSB2, ZmTSB1, ZmIGS, AUX2, TAR2, Yucca6, and AGPLS3, followed by selection.

As used herein, the term “(novel) germplasm” refers to maize plants or progeny materials obtained by gene editing according to the present application and suitable for use in breeding.

Beneficial technical effects of one or more of the above technical solutions include:
starting from improving the efficiency of the technology itself, the present invention selects multiple functional genes related to starch synthesis and ear rot, including studied genes related to starch content and ear rot: ZmGBSSI, Shrunken 2, AGPLS3, Brittle 2, ZmDOF36, ZmTPS9, ZmAuxRP1, ZmSIZ1a, ZmSIZ1b, and ZmFER1, and genes predicted to be related: ZmTAR4-1, ZmTAR4-3, Yucca2, Yucca4, ZmTSB2, ZmTSB1, ZmIGS, ZmAUX2, ZmTAR2, and ZmYUC6. By using high-throughput gene editing technology to efficiently and randomly aggregate numerous advantageous loci, and by adding appropriate concentrations of betaine and calcium chloride during genetic transformation, more loci are edited in a single transgenic event with better efficiency. This effectively improves diversity and generates more superior locus combination patterns, and efficiently and at scale generates numerous novel variant germplasm having high starch content and low mycotoxin content (e.g., resistance to ear rot and other diseases), representing an efficient application method for using gene editing to generate novel germplasm needed for breeding. Meanwhile, by conducting multi-location and multi-replicate field phenotypic identification, the present invention enables large-scale, systematic, and precise screening of superior planting resources required for breeding, and allows rapid and efficient application in breeding.

### BRIEF DESCRIPTION OF DRAWINGS

The accompanying drawings, which form a part of this specification, provide a further understanding of the present invention. The schematic embodiments of the present invention and the descriptions thereof are used to explain the present invention and do not constitute improper limitation of the present invention.

FIG. 1 shows, in the Examples of the present invention, the average number of edits per single event among transformation events obtained by betaine and calcium chloride treatment, wherein the betaine concentration is 100 mM and the calcium chloride concentration is 50 mM.

FIG. 2 shows, in the Examples of the present invention, high-starch mutants identified and selected from gene-edited materials and the starch contents thereof, wherein zml-cr11 and zml-cr22 were selected from transformation events obtained by betaine treatment; zml-cr42 and zml-cr56 were selected from transformation events obtained by calcium chloride treatment; and zml-cr74 and zml-cr92 were selected from transformation events obtained by combined betaine+calcium chloride treatment.

### DESCRIPTION OF THE INVENTION

It should be noted that the following detailed description is exemplary and is intended to provide further explanation of the present invention. Unless otherwise indicated, all technical and scientific terms used herein have the same meanings as commonly understood by one of ordinary skill in the art to which the present invention belongs.

It should be understood that the terminology used herein is for the purpose of describing particular embodiments only and is not intended to limit the exemplary embodiments of the present invention. As used herein, unless otherwise clearly indicated by the context, the singular forms are intended to include the plural forms as well. In addition, it should be understood that when the terms “comprise” and/or “include” are used in this specification, they specify the presence of stated features, steps, operations, devices, components, and/or combinations thereof. The scope of the present invention is not limited to the specific embodiments described below, and the terminology used in the embodiments is intended to describe specific embodiments rather than to limit the scope of the present invention.

The embodiments of the present application will be described in detail below with reference to Examples. It should be understood that these Examples are only for illustrating the present application and are not intended to limit the scope of the present application. For experimental methods in the following Examples for which specific conditions are not indicated, priority is given to the guidance provided in the present application; alternatively, the methods may be performed according to laboratory manuals or conventional conditions in the art, according to conditions recommended by the manufacturer, or with reference to known experimental methods in the art.

In the specific Examples below, measurement parameters of raw material components may have slight deviations within the weighing accuracy range unless otherwise specified. For temperature and time parameters, acceptable deviations caused by instrument testing accuracy or operational accuracy are allowed.

### Definitions

As used in the present invention, “multi-gene editing technology” refers to a technology that uses CRISPR/Cas9 gene editing to simultaneously edit multiple loci in a single transgenic event.

As used in the present invention, “gene editing efficiency” refers to aggregated editing efficiency, i.e., the number of vectors corresponding to multiple target loci edits occurring in one transgenic event using CRISPR/Cas9-mediated multi-gene editing technology.

As used in the present invention, a “target locus” refers to a target site.

### Example 1

A Method for Efficiently Producing Novel Germplasm with Low Mycotoxin Content and High Starch Content Using Gene Editing Technology

### I. Experimental Materials

1. Immature Embryos of Maize (Zea mays) Inbred Line KN5585.

The backbone vector for gene editing targets was pCXB053, and the Agrobacterium tumefaciens strain was EHA105 (purchased from Shanghai WeidiBio).
Test kits for measuring kernel starch content and mycotoxin content.
Media: YP medium, infection medium, co-cultivation medium, resting medium, selection medium 1, selection medium 2, differentiation medium, and rooting medium. The specific compositions are shown in Table 1.

TABLE 1
Compositions of Different MS Media

| MS Medium Type | Medium Components |
|---|---|
| YP medium | NaCl 5 g/L; yeast extract 5 g/L; peptone 10 |
| | g/L; agar 15 g/L |
| Infection medium | ½ MS; sucrose 68.5 g/L; glucose 36 g/L; L- |
| | proline 0.115 g/L; acetosyringone 200 mM; |
| | cysteine 200 mg/L; mixed Agrobacterium |
| | suspension at OD600 of 0.5-1; and at least |
| | one selected from betaine 100 mM, calcium |
| | chloride 50 mM, and a combination thereof |
| Co-cultivation | ½ MS; sucrose 20 g/L; glucose 10 g/L; |
| medium | proline 0.115 g/L; thiamine hydrochloride 0.5 |
| | mg/L; AgNO₃ 20 mM; L-cysteine 200 mg/L; |
| | 2,4-D 0.5 mg/L; dicamba 2.2 mg/L; |
| | acetosyringone 200 mM |
| Resting medium | MS; sucrose 30 g/L; proline 1.38 g/L; thiamine |
| | hydrochloride 0.5 mg/L; AgNO₃ 20 mM; |
| | casein hydrolysate 0.5 g/L; 2,4-D 0.5 mg/L; |
| | dicamba 2.2 mg/L; timentin 200 mg/L |
| Selection medium 1 | MS; sucrose 30 g/L; proline 1.38 g/L; thiamine |
| | hydrochloride 0.5 mg/L; AgNO₃ 20 mM; |
| | casein hydrolysate 0.5 g/L; 2,4-D 0.5 mg/L; |
| | bialaphos 5 mg/L |
| Selection medium 2 | MS; sucrose 30 g/L; proline 1.38 g/L; thiamine |
| | hydrochloride 0.5 mg/L; AgNO₃ 20 mM; |
| | casein hydrolysate 0.5 g/L; 2,4-D 0.5 mg/L; |
| | bialaphos 8 mg/L |
| Differentiation | MS; sucrose 20 g/L; 6-BA 0.1 mg/L; KT 1 |
| medium | mg/L; timentin 200 mg/L |
| Rooting medium | MS; sucrose 20 g/L; MES 0.5 g/L; IBA 0.2 mg/L |

The above trace components were first prepared as stock solutions. Bulk solid components were weighed using a balance and fully dissolved in an appropriate amount of deionized water according to the target system; the pH was adjusted; the volume was brought up to the final volume; and the mixture was sterilized by autoclaving for 20 min under high temperature and high pressure. After cooling to about 50° C., heat-labile liquid components were sterilized by filtration and added to the medium using a disposable syringe. The medium was poured into Petri dishes and stored at 4° C. for later use.

### II. Experimental Methods

1. Selection of Target Genes and gRNA Design
1.1 Gene selection: a total of 20 genes related to increased starch content and low mycotoxin content (ear rot-related genes) were selected, including 6 starch-related genes and 14 ear rot-related genes. Studies have shown that decreased auxin content in ears is beneficial for improving maize resistance to ear rot disease; therefore, genes affecting auxin content were also selected as pre-editing genes of the present invention, including Yucca2, Yucca4, Yucca6, TAR4-1, TAR4-3, AUX2, TAR2, ZmIGS, ZmTSB1, and ZmTSB2. The gene names and gene IDs are shown in Table 2.

TABLE 2
Gene Names and Gene IDs

| Gene Name | Gene ID |
|---|---|
| ZmGBSSI | GRMZM2G024993/Zm00001eb378140 |
| Shrunken 2 | GRMZM2G429899/Zm00001eb159060 |
| Brittle 2 | GRMZM2G068506/Zm00001eb176800 |
| ZmDOF36 | GRMZM2G137502_T02/Zm00001eb020000 |
| ZmTPS9 | GRMZM2G312521/Zm00001eb192680/Zm00001d052060 |
| ZmAuxRP1 | GRMZM2G063298/Zm00001eb053610 |
| TAR4-1 | GRMZM2G054115/Zm00001eb154790 |
| TAR4-3 | GRMZM2G127308/Zm00001eb336530/Zm00001d008700 |
| | (V4) |
| Yucca2 | GRMZM2G159393/Zm00001eb419450/Zm00001d025005 |
| Yucca4 | GRMZM2G141383/Zm00001eb088950 |
| ZmSIZ1a | GRMZM2G155123/Zm00001eb355110/Zm00001d010974 |
| ZmSIZ1b | GRMZM2G337659/Zm00001eb265410/Zm00001d035487 |
| ZmFER1 | GRMZM2G180622/Zm00001eb126340/Zm00001d040118 |
| ZmTSB2 | GRMZM2G054465/Zm00001eb387730/Zm00001d046676 |
| ZmTSB1 | GRMZM2G169593/Zm00001eb173100/Zm00001d049610 |
| ZmIGS | GRMZM2G106950/Zm00001eb309260/Zm00001d020008 |
| AUX2 | GRMZM2G156470/Zm00001eb104170/Zm00001d006283 |
| TAR2 | GRMZM2G066345/Zm00001eb123040/Zm00001d039691 |
| Yucca6 | GRMZM2G019515/Zm00001eb332870/Zm00001d008255 |
| AGPLS3 | GRMZM2G144002/Zm00001eb304130 |

1.2 Batch target synthesis: batch target synthesis was performed using a dual-target strategy for each vector. The genes were classified into negative regulators and positive regulators. For 13 negatively regulated genes, targets were designed in coding regions to introduce frameshift mutations, with 2 target sites designed for each gene, and 13 dual-target vectors were constructed, with the expectation of obtaining variant germplasm with partial or complete loss of gene function. The 3′ UTR (3′ untranslated region) is a non-translated region at the tail end of a transcript and plays an important role in post-transcriptional regulation; it has a significant impact on regulation of gene expression, mRNA stability, localization, and post-transcriptional modification. For 7 positively regulated genes, a dual-target strategy was adopted in the 3′ UTR region to delete fragments. For each gene, two pairs (four targets) of combinations were designed, and two dual-vector constructs were constructed per gene, for a total of 14 vectors, with the expectation of obtaining variant germplasm with upregulated gene protein expression. In total, 27 dual-target vectors were required, and novel germplasm materials with high starch content and low mycotoxin content were expected to be generated. The specific primers and sequences are shown in Table 3.

TABLE 3
Primers and Sequences

| | Dual- | | |
|---|---|---|---|
| Gene | target | | Oligonucleotides |
| name | vector | Target sequence | corresponding to target sites |
| ZmGBSSI | ZmGBSSI- | sgRNA1: GGCCCCCTGATCTCGCGC | SG1F: ATTGGGCCCCCTGATCTCGCGCGGTTT |
| | a | G (SEQ ID NO: 1) | (SEQ ID NO: 54) |
| | | | SG1R: TCTAAAACCGCGCGAGATCAGGGGG |
| | | | CC (SEQ ID NO: 55) |
| | | sgRNA2: GCAATATAAAATTACCGA | SG2F: ACCGAATTGGCAATATAAAATTACCG |
| | | T (SEQ ID NO: 2) | AT (SEQ ID NO: 56) |
| | | | SG2R: AAACATCGGTAATTTTATATTGCCAAT |
| | | | T (SEQ ID NO: 57) |
| | ZmGBSSI- | sgRNA1: CACCACGCGCGAGATCA | SG1F: ATTGCACCACGCGCGAGATCAGGGTT |
| | b | GG (SEQ ID NO: 3) | T (SEQ ID NO: 58) |
| | | | SG1R: TCTAAAACGGCCCCCTGATCTCGCGC |
| | | | G (SEQ ID NO: 59) |
| | | sgRNA2: ATAAGAACACCGAACAG | SG2F: ACCGAATTGATAAGAACACCGAACAG |
| | | CA (SEQ ID NO: 4) | CA (SEQ ID NO: 60) |
| | | | SG2R: AAACTGCTGTTCGGTGTTCTTATCAAT |
| | | | T (SEQ ID NO: 61) |
| ZmSH2 | SH2-a | sgRNA1: ACAAAACAAGAACCTACA | SG1F: ATTGACAAAACAAGAACCTACAAGTT |
| | | A (SEQ ID NO: 5) | T (SEQ ID NO: 62) |
| | | | SG1R: TCTAAAACTTGTAGGTTCTTGTTTTGT |
| | | | (SEQ ID NO: 63) |
| | | sgRNA2: TACATACTAGCAGCACAA | SG2F: ACCGAATTGTACATACTAGCAGCACA |
| | | A (SEQ ID NO: 6) | AA (SEQ ID NO: 64) |
| | | | SG2R: AAACTTTGTGCTGCTAGTATGTACAAT |
| | | | T (SEQ ID NO: 65) |
| | SH2-b | sgRNA1: GGATCGTGTAACCTTGGT | SG1F: ATTGGGATCGTGTAACCTTGGTAGTTT |
| | | A (SEQ ID NO: 7) | (SEQ ID NO: 66) |
| | | | SG1R: TCTAAAACGGATCGTGTAACCTTGGT |
| | | | A (SEQ ID NO: 67) |
| | | sgRNA2: GTTGATCCAAAGCAGGTA | SG2F: ACCGAATTGGTTGATCCAAAGCAGGT |
| | | A (SEQ ID NO: 8) | AA (SEQ ID NO: 68) |
| | | | SG2R: AAACTTACCTGCTTTGGATCAACCAAT |
| | | | T (SEQ ID NO: 69) |
| ZmAGPLS3 | AGPLS3- | sgRNA1: TTTCAAAGCAGCAACCAC | SG1F: ATTGTTTCAAAGCAGCAACCACAGTTT |
| | a | A (SEQ ID NO: 9) | (SEQ ID NO: 70) |
| | | | SG1R: TCTAAAACTGTGGTTGCTGCTTTGAAA |
| | | | (SEQ ID NO: 71) |
| | | sgRNA2: TACATGTAGAGGGAGAG | SG2F: ACCGAATTGTACATGTAGAGGGAGAG |
| | | AT (SEQ ID NO: 10) | AT (SEQ ID NO: 72) |
| | | | SG2R: AAACATCTCTCCCTCTACATGTACAAT |
| | | | T (SEQ ID NO: 73) |
| | AGPLS3- | sgRNA1: AGCACCTGCAACACGCCA | SG1F: ATTGAGCACCTGCAACACGCCAAGTT |
| | b | A (SEQ ID NO: 11) | T (SEQ ID NO: 74) |
| | | | SG1R: TCTAAAACTTGGCGTGTTGCAGGTGC |
| | | | T (SEQ ID NO: 75) |
| | | sgRNA2: ACTTGGCACATGAGATGC | SG2F: ACCGAATTGACTTGGCACATGAGATG |
| | | A (SEQ ID NO: 12) | CA (SEQ ID NO: 76) |
| | | | SG2R: AAACTGCATCTCATGTGCCAAGTCAA |
| | | | TT (SEQ ID NO: 77) |
| Bt2 | Bt2-a | sgRNA1: TGTGTCCTTCAATGTTGT | SG1F: ATTGTGTGTCCTTCAATGTTGTAGTTT |
| | | A (SEQ ID NO: 13) | (SEQ ID NO: 78) |
| | | | SG1R: TCTAAAACTACAACATTGAAGGACAC |
| | | | A (SEQ ID NO: 79) |
| | | sgRNA2: TCTAACACATAGGATGCG | SG2F: ACCGAATTGTCTAACACATAGGATGC |
| | | G (SEQ ID NO: 14) | GG (SEQ ID NO: 80) |
| | | | SG2R: AAACCCGCATCCTATGTGTTAGACAA |
| | | | TT (SEQ ID NO: 81) |
| | Bt2-b | sgRNA1: TATTCGTCAGGCACAAAA | SG1F: ATTGTATTCGTCAGGCACAAAAGGTTT |
| | | G (SEQ ID NO: 15) | (SEQ ID NO: 82) |
| | | | SG1R: TCTAAAACCTTTTGTGCCTGACGAATA |
| | | | (SEQ ID NO: 83) |
| | | sgRNA2: CAATCCACTCCATTCTACA | SG2F: ACCGAATTGCAATCCACTCCATTCTAC |
| | | (SEQ ID NO: 16) | A (SEQ ID NO: 84) |
| | | | SG2R: AAACTGTAGAATGGAGTGGATTGCAA |
| | | | TT (SEQ ID NO: 85) |
| ZmDOF36 | DOF36-a | sgRNA1: CACCGACCAGATGATGC | SG1F: ATTGCACCGACCAGATGATGCGAGTT |
| | | GA (SEQ ID NO: 17) | T (SEQ ID NO: 86) |
| | | | SG1R: TCTAAAACTCGCATCATCTGGTCGGT |
| | | | G (SEQ ID NO: 87) |
| | | sgRNA2: TTAACAAAGAAATAAACT | SG2F: ACCGAATTGTTAACAAAGAAATAAAC |
| | | G (SEQ ID NO: 18) | TG (SEQ ID NO: 88) |
| | | | SG2R: AAACCAGTTTATTTCTTTGTTAACAAT |
| | | | T (SEQ ID NO:89) |
| | DOF36-b | sgRNA1: CTCCCTCGCATCATCTGG | SG1F: ATTGCTCCCTCGCATCATCTGGTGTTT |
| | | T (SEQ ID NO: 19) | (SEQ ID NO: 90) |
| | | | SG1R: TCTAAAACACCAGATGATGCGAGGGA |
| | | | G (SEQ ID NO: 91) |
| | | sgRNA2: CATCAAGCAGATATAACG | SG2F: ACCGAATTGCATCAAGCAGATATAAC |
| | | C (SEQ ID NO: 20) | GC (SEQ ID NO: 92) |
| | | | SG2R: AAACGCGTTATATCTGCTTGATGCAAT |
| | | | T (SEQ ID NO: 93) |
| ZmTPS9 | TPS9-a | sgRNA1: CGGAGAACCTGAATCCG | SG1F: ATTGCGGAGAACCTGAATCCGAAGTT |
| | | AA (SEQ ID NO: 21) | T (SEQ ID NO: 94) |
| | | | SG1R: TCTAAAACTTCGGATTCAGGTTCTCCG |
| | | | (SEQ ID NO: 95) |
| | | sgRNA2: TATCTTGACAGTCACGGT | SG2F: ACCGAATTGTATCTTGACAGTCACGGT |
| | | G (SEQ ID NO: 22) | G (SEQ ID NO: 96) |
| | | | SG2R: AAACCACCGTGACTGTCAAGATACAA |
| | | | TT (SEQ ID NO: 97) |
| ZmAuxRP1 | AuxRP1- | sgRNA1: ATCACCTGAACATGGTGC | SG1F: ATTGATCACCTGAACATGGTGCGGTTT |
| | a | G (SEQ ID NO: 23) | (SEQ ID NO: 98) |
| | | | SG1R: TCTAAAACCGCACCATGTTCAGGTGA |
| | | | T (SEQ ID NO: 99) |
| | | sgRNA2: CAGAGCCACCCTCGCCGG | SG2F: ACCGAATTGCAGAGCCACCCTCGCCG |
| | | T (SEQ ID NO: 24) | GT (SEQ ID NO: 100) |
| | | | SG2R: AAACACCGGCGAGGGTGGCTCTGCA |
| | | | ATT (SEQ ID NO: 101) |
| ZmTAR4-1 | TAR4-1- | sgRNA1: TACAGATGGGCCGGAAC | SG1F: ATTGTACAGATGGGCCGGAACCAGTT |
| | a | CA (SEQ ID NO: 25) | T (SEQ ID NO: 102) |
| | | | SG1R: TCTAAAACTGGTTCCGGCCCATCTGTA |
| | | | (SEQ ID NO: 103) |
| | | sgRNA2: GAGAAGCGCGTATGCCT | SG2F: ACCGAATTGGAGAAGCGCGTATGCCT |
| | | GT (SEQ ID NO: 26) | GT (SEQ ID NO: 104) |
| | | | SG2R: AAACACAGGCATACGCGCTTCTCCAA |
| | | | TT (SEQ ID NO: 105) |
| ZmTAA1 | TAA1-a | sgRNA1: TTAGCGGTGAGGAGGCG | SG1F: ATTGTTAGCGGTGAGGAGGCGCGGTT |
| | | CG (SEQ ID NO: 27) | T (SEQ ID NO: 106) |
| | | | SG1R: TCTAAAACCGCGCCTCCTCACCGCTAA |
| | | | (SEQ ID NO: 107) |
| | | sgRNA2: GGCCCACCAAAAGGCCG | SG2F: ACCGAATTGGGCCCACCAAAAGGCCG |
| | | CG (SEQ ID NO: 28) | CG (SEQ ID NO:108) |
| | | | SG2R: AAACCGCGGCCTTTTGGTGGGCCCAA |
| | | | TT (SEQ ID NO: 109) |
| Yucca2 | Yucca2-a | sgRNA1: TAGCGCGCCAACACCACC | SG1F: ATTGTAGCGCGCCAACACCACCGGTT |
| | | G (SEQ ID NO: 29) | T (SEQ ID NO: 110) |
| | | | SG1R: TCTAAAACCGGTGGTGTTGGCGCGCT |
| | | | A (SEQ ID NO: 111) |
| | | sgRNA2: TCGAGCTCAAGGAGACA | SG2F: ACCGAATTGTCGAGCTCAAGGAGACA |
| | | CA (SEQ ID NO: 30) | CA (SEQ ID NO: 112) |
| | | | SG2R: AAACTGTGTCTCCTTGAGCTCGACAAT |
| | | | T (SEQ ID NO: 113) |
| Yucca4 | Yucca4-a | sgRNA1: GGAGAAGAGGCTATCCA | SG1F: ATTGGGAGAAGAGGCTATCCATGGTT |
| | | TG (SEQ ID NO: 31) | T (SEQ ID NO: 114) |
| | | | SG1R: TCTAAAACCATGGATAGCCTCTTCTCC |
| | | | (SEQ ID NO: 115) |
| | | sgRNA2: GACTTGTACTCGCTCACG | SG2F: ACCGAATTGGACTTGTACTCGCTCACG |
| | | T (SEQ ID NO: 32) | T (SEQ ID NO: 116) |
| | | | SG2R: AAACACGTGAGCGAGTACAAGTCCAA |
| | | | TT (SEQ ID NO: 117) |
| ZmSIZ1a | SIZ1a-a | sgRNA1: GTGTTCTATATGATCCAA | SG1F: ATTGGTGTTCTATATGATCCAAGGTTT |
| | | G (SEQ ID NO: 33) | (SEQ ID NO: 118) |
| | | | SG1R: TCTAAAACCTTGGATCATATAGAACA |
| | | | C (SEQ ID NO: 119) |
| | | sgRNA2: ATCAGAGAAGAATAGCG | SG2F: ACCGAATTGATCAGAGAAGAATAGCG |
| | | AG (SEQ ID NO: 34) | AG (SEQ ID NO: 120) |
| | | | SG2R: AAACACTCGCTATTCTTCTCTGATCAA |
| | | | TT (SEQ ID NO: 121) |
| | SIZ1a-b | sgRNA1: AACATATCAGTTACCACG | SG1F: ATTGAACATATCAGTTACCACGTGTTT |
| | | T (SEQ ID NO: 35) | (SEQ ID NO: 122) |
| | | | SG1R: TCTAAAACACGTGGTAACTGATATGT |
| | | | T (SEQ ID NO: 123) |
| | | sgRNA2: TGTCTTGATGCCAGCTGA | SG2F: ACCGAATTGTGTCTTGATGCCAGCTG |
| | | T (SEQ ID NO: 36) | AT (SEQ ID NO: 124) |
| | | | SG2R: AAACATCAGCTGGCATCAAGACACAA |
| | | | TT (SEQ ID NO: 125) |
| ZmSIZ1b | SIZ1b-a | sgRNA1: TCTTGATCGTTACTGTGG | SG1F: ATTGTCTTGATCGTTACTGTGGAGTTT |
| | | A (SEQ ID NO: 37) | (SEQ ID NO: 126) |
| | | | SG1R: TCTAAAACTCCACAGTAACGATCAAG |
| | | | A (SEQ ID NO: 127) |
| | | sgRNA2: GTGTTCTATATGATCCAA | SG2F: ACCGAATTGGTGTTCTATATGATCCAA |
| | | G (SEQ ID NO: 33) | G (SEQ ID NO: 128) |
| | | | SG2R: AAACCTTGGATCATATAGAACACCAA |
| | | | TT (SEQ ID NO: 129) |
| | SIZ1b-b | sgRNA1: CGCAACAAGAACAGATG | SG1F: ATTGCGCAACAAGAACAGATGAAGTT |
| | | AA (SEQ ID NO: 38) | T (SEQ ID NO: 130) |
| | | | SG1R: TCTAAAACTTCATCTGTTCTTGTTGCG |
| | | | (SEQ ID NO: 131) |
| | | sgRNA2: CCAGCGCCTTTCAATCGC | SG2F: ACCGAATTGCCAGCGCCTTTCAATCGC |
| | | C (SEQ ID NO: 39) | C (SEQ ID NO: 132) |
| | | | SG2R: AAACGGCGATTGAAAGGCGCTGGCA |
| | | | ATT (SEQ ID NO: 133) |
| ZmFER1 | FER1-a | sgRNA1: GTCGAGGAGAAGAAAAG | SG1F: ATTGGTCGAGGAGAAGAAAAGAAGT |
| | | AA (SEQ ID NO: 40) | TT (SEQ ID NO: 134) |
| | | | SG1R: TCTAAAACTTCTTTTCTTCTCCTCGAC |
| | | | (SEQ ID NO: 135) |
| | | sgRNA2: GAAACAGCAGCTAAGGC | SG2F: ACCGAATTGGAAACAGCAGCTAAGGC |
| | | CA (SEQ ID NO: 41) | CA (SEQ ID NO: 136) |
| | | | SG2R: AAACTGGCCTTAGCTGCTGTTTCCAAT |
| | | | T (SEQ ID NO: 137) |
| ZmTSB2 | TSB2-a | sgRNA1: CAGGCGTGGTACAACGC | SG1F: ATTGCAGGCGTGGTACAACGCCGGTT |
| | | CG (SEQ ID NO: 42) | T (SEQ ID NO: 138) |
| | | | SG1R: TCTAAAACCGGCGTTGTACCACGCCT |
| | | | G (SEQ ID NO: 139) |
| | | sgRNA2: GGCACTTCGATTCGCAGG | SG2F: ACCGAATTGGGCACTTCGATTCGCAG |
| | | T (SEQ ID NO: 43) | GT (SEQ ID NO: 140) |
| | | | SG2R: AAACACCTGCGAATCGAAGTGCCCAA |
| | | | TT (SEQ ID NO: 141) |
| ZmTSB1 | TSB1-a | sgRNA1: CTCATCTACCTCAAGAGG | SG1F: ATTGCTCATCTACCTCAAGAGGGGTTT |
| | | G (SEQ ID NO: 44) | (SEQ ID NO: 142) |
| | | | SG1R: TCTAAAACCCCTCTTGAGGTAGATGA |
| | | | G (SEQ ID NO: 143) |
| | | sgRNA2: GAGATGGGGAACGGCGC | SG2F: ACCGAATTGGAGATGGGGAACGGCG |
| | | CG (SEQ ID NO: 45) | CCG (SEQ ID NO: 144) |
| | | | SG2R: AAACCGGCGCCGTTCCCCATCTCCAAT |
| | | | T (SEQ ID NO: 145) |
| ZmIGS | IGS-a | sgRNA1: ACGCCTTCACCTCGCACA | SG1F: ATTGACGCCTTCACCTCGCACAGGTTT |
| | | G (SEQ ID NO: 46) | (SEQ ID NO: 146) |
| | | | SG1R: TCTAAAACCTGTGCGAGGTGAAGGCG |
| | | | T (SEQ ID NO: 147) |
| | | sgRNA2: CTCGCACCTACGAGAAGA | SG2F: ACCGAATTGCTCGCACCTACGAGAAG |
| | | A (SEQ ID NO: 47) | AA (SEQ ID NO: 148) |
| | | | SG2R: AAACTTCTTCTCGTAGGTGCGAGCAA |
| | | | TT (SEQ ID NO: 149) |
| ZmAUX2 | AUX2-a | sgRNA1: GCGAGCAAGGGCCATTG | SG1F: ATTGGCGAGCAAGGGCCATTGCGGTT |
| | | CG (SEQ ID NO: 48) | T (SEQ ID NO: 150) |
| | | | SG1R: TCTAAAACCGCAATGGCCCTTGCTCG |
| | | | C (SEQ ID NO: 151) |
| | | sgRNA2: GCGACATCAGCAAATGG | SG2F: ACCGAATTGGCGACATCAGCAAATGG |
| | | AG (SEQ ID NO: 49) | AG (SEQ ID NO: 152) |
| | | | SG2R: AAACCTCCATTTGCTGATGTCGCCAAT |
| | | | T (SEQ ID NO: 153) |
| ZmTAR2 | TAR2-a | sgRNA1: CAGCACTACGTCAGTAGC | SG1F: ATTGCAGCACTACGTCAGTAGCAGTTT |
| | | A (SEQ ID NO: 50) | (SEQ ID NO: 154) |
| | | | SG1R: TCTAAAACTGCTACTGACGTAGTGCT |
| | | | G (SEQ ID NO: 155) |
| | | sgRNA2: CAGGCGCAGAGTCGTCG | SG2F: ACCGAATTGCAGGCGCAGAGTCGTCG |
| | | GG (SEQ ID NO: 51) | GG (SEQ ID NO: 156) |
| | | | SG2R: AAACCCCGACGACTCTGCGCCTGCAA |
| | | | TT (SEQ ID NO: 157) |
| ZmYUC6 | YUC6-a | sgRNA1: TACGCGGCGAGGTTCCTG | SG1F: ATTGTACGCGGCGAGGTTCCTGGGTT |
| | | G (SEQ ID NO: 52) | T (SEQ ID NO: 158) |
| | | | SG1R: TCTAAAACCCAGGAACCTCGCCGCGT |
| | | | A (SEQ ID NO: 159) |
| | | sgRNA2: CACGCAGTGGAGTACCG | SG2F: ACCGAATTGCACGCAGTGGAGTACCG |
| | | GT (SEQ ID NO: 53) | GT (SEQ ID NO: 160) |
| | | | SG2R: AAACACCGGTACTCCACTGCGTGCAA |
| | | | TT (SEQ ID NO: 161) |

1.3 High-throughput vector construction

Specific sgRNA sequences were designed and screened for target genes using CRISPR-P 2.0 software. After adding the restriction enzyme site BsaI, the sequences were used to synthesize primers for subsequent preparation of dual-target vectors. The vector construction method refers to Zhang Maolin, Wang Juan, Yan Jiali, et al., “Generating diversified allelic variations in maize using CRISPR/Cas9-mediated fragment deletion technology” (Shandong Agricultural Sciences, 2024, 56 (09): 1-5; DOI: 10.14083/j.issn.1001-4942.2024.09.001). The backbone vector used for targets was pCXB053 (see, e.g., DOI: 10.1105/tpc.19.00934). Dual-target vectors were retained for use after sequencing confirmed correct construction.

### 2. Preparation and Pooling of Agrobacterium

The constructed plasmids were individually transformed into 100 μL of Agrobacterium EHA105. Successfully transformed Agrobacterium were individually activated in YP liquid medium. When the OD600 reached the same value between 0.5 and 1 (OD600 of 0.6 was used in this experiment), 100 μL of each culture was taken and mixed to prepare a pooled Agrobacterium culture for subsequent genetic transformation.

### 3. High-Throughput Genetic Transformation and Selection

The pooled Agrobacterium containing the plasmids was used to transform the recipient inbred line KN5585. During genetic transformation, the infection medium was treated with betaine and/or calcium chloride. Treatment 1:100 mM betaine; Treatment 2:50 mM calcium chloride; Treatment 3:100 mM betaine+50 mM calcium chloride. A normal AS suspension without betaine and calcium chloride was used as a control. The treatments are shown in Table 4.

TABLE 4
Infection Medium Treatment Methods

| Control | AS-containing suspension + Agrobacterium |
|---|---|
| Treatment 1 | AS-containing suspension + 100 mM betaine + Agrobacterium |
| Treatment 2 | AS-containing suspension + 50 mM calcium chloride + Agrobacterium |
| Treatment 3 | AS-containing suspension + 100 mM betaine + 50 mM calcium |
| | chloride + Agrobacterium |

In the above, the suspension was: ½ MS+sucrose 68.5 g/L+glucose 36 g/L+L-proline 0.115 g/L.

The AS-containing suspension was: ½ MS+sucrose 68.5 g/L+glucose 36 g/L+L-proline 0.115 g/L+acetosyringone (AS) 200 mM+cysteine 200 mg/L.

Specifically, immature embryos of about 1 mm in size from maize inbred line KN5585 (an inbred line bred by Weimi Biotechnology (Jiangsu) Co., Ltd.) were freshly excised and used as materials. The maize embryos were placed into a 2 mL plastic centrifuge tube containing 1.8 mL of a suspension or treatment solution (Table 4) to maintain osmotic pressure. Within 30 min, approximately 150 immature embryos were treated. The suspension was removed, and 1.0 ml of infection medium containing Agrobacterium was added to the tube containing the embryos, followed by standing for 5 min. The Agrobacterium concentration in the infection medium was diluted to an OD600 of 0.5-1; the OD600 of 0.6 was used in this experiment.

The embryos were resuspended in the centrifuge tube and poured onto a co-cultivation medium, and excess Agrobacterium suspension on the surface was removed with a pipette. The embryos were co-cultured in the dark at 23° C. for 3 days. After co-cultivation, the embryos were transferred to a resting medium and cultured in the dark at 28° C. for 6 days. The embryos were then placed on selection medium 1 containing 5 mg/L bialaphos (a herbicide) for selection culture for 2 weeks, and then transferred to selection medium 2 containing 8 mg/L bialaphos for selection culture for 2 weeks to obtain resistant callus.

The resistant callus was transferred to a differentiation medium and cultured at 25° C. under light at 5,000 lux for 3 weeks. Seedlings generated by differentiation were transferred to a rooting medium and cultured at 25° C. under light at 5,000 lux until rooting. The seedlings were then transferred to small pots for growth, and after reaching a certain growth stage, transplanted into a greenhouse. Progeny seeds were harvested after 3-4 months.

### 4. Identification of Editing Types and Calculation of Aggregated Editing Efficiency

4.1 The obtained positive seedlings were planted in a modern greenhouse in Xishuangbanna, carefully maintained and managed, and seeds were harvested. Homozygous transformation events were obtained by segregation to remove the transgene system. No fewer than 80 homozygous transformation events were obtained for each treatment.
4.2 Leaves from experimental materials grown in the field were collected and DNA was extracted. Meanwhile, 23 pairs of primers were designed according to the target editing positions to identify editing types. The primers used are shown in Table 5.

TABLE 5
Identification Primer Sequences

| Gene | Detection Primer F | Detection Primer R |
|---|---|---|
| ZmGBSSI | ZmGBSSI-F: | ZmGBSSI-R: |
| | CAGTGCAACGTCGTGGAGC (SEQ ID | CGTAGGATTCAGAAAGAAAATG |
| | NO: 162) | (SEQ ID NO: 163) |
| ZmSH2 | ZmSH2-F: | ZmSH2-R: |
| | GTGACAGGGCATCCAAGAGG (SEQ | CATCGAAGCCAGATCCCAAAAG |
| | ID NO: 164) | (SEQ ID NO: 165) |
| ZmAGPLS3 | ZmAGPLS3-F: | ZmAGPLS3-R: |
| | CCTGCTACCTACAGTTCCTGG (SEQ | GCATCAATAGGCAATTAGG (SEQ ID |
| | ID NO: 166) | NO: 167) |
| Bt2 | Bt2-F: CTGTTGGAGATCATCGGAG | Bt2-R: CCCTAACCCTAATCCTCTG (SEQ |
| | (SEQ ID NO: 168) | ID NO: 169) |
| ZmDOF36 | ZmDOF36-F: | ZmDOF36-R: |
| | CAGTGCCAGCCACAGGAGG (SEQ ID | GCACAGAGTTTGTAAATGC (SEQ ID |
| | NO: 170) | NO: 171) |
| ZmTPS9 | ZmTPS9-F: | ZmTPS9-R: |
| | GTCATCGTCTCCCACCGTCTG (SEQ | CCATCGCCAGGAACTTGAG (SEQ ID |
| | ID NO: 172) | NO: 173) |
| ZmAuxRP1 | ZmAuxRP1-F: | ZmAuxRP1-R: |
| | GACGATGGCTTATTITCTGCTC (SEQ | CTGTCCGTCATCACGTTGTCC (SEQ |
| | ID NO: 174) | ID NO: 175) |
| ZmTAR4-1 | ZmTAR4-1-F: | ZmTAR4-1-R: |
| | GGTGCGAGTGCAACAGCTG (SEQ ID | GATGGCTTGGAGACGGTGAAC |
| | NO: 176) | (SEQ ID NO: 177) |
| ZmTAA1 | ZmTAA1-F: | ZmTAA1-R: |
| | CACGAGCAGGCTGCTGTAGG (SEQ | GTGCGACGGAGCTGTGCTGTTG |
| | ID NO: 178) | (SEQ ID NO: 179) |
| Yucca2 | Yucca2-F: CGCATGGACAGCCTCTTCTC | Yucca2-R: |
| | (SEQ ID NO: 180) | CAGGGACGACGGCGATATCTC (SEQ |
| | | ID NO: 181) |
| Yucca4 | Yucca4-F: | Yucca4-R: |
| | CACCTTCTCCTCCTCATTCTTC (SEQ | CAGGTCCAGCGACACCTCCATC |
| | ID NO: 182) | (SEQ ID NO: 183) |
| ZmSIZ1a | ZmSIZ1a-F1: | ZmSIZ1a-R1: |
| | GATTCAGAGTAGTTCGGACATC | CAGCCACCTCCTATCTCATC (SEQ |
| | (SEQ ID NO: 184) | ID NO: 185) |
| ZmSIZ1a | ZmSIZ1a-F2: | ZmSIZ1a-R2: |
| | CGATGTGAGTGCCTGTAGTTTC | GCAACCTGCATTCTACTGTTAG |
| | (SEQ ID NO: 186) | (SEQ ID NO: 187) |
| ZmSIZ1b | ZmSIZ1b-F1: | ZmSIZ1b-R1: |
| | CACTGGGATAGCTGAATTTG (SEQ | CATCTCCAATGCCGTGTTC (SEQ ID |
| | ID NO: 188) | NO: 189) |
| ZmSIZ1b | ZmSIZ1b-F2: | ZmSIZ1b-R2: |
| | GTATTTAGGGTTTGGGTGC (SEQ ID | CTTTAACAACCTGTCTTCTACC |
| | NO: 190) | (SEQ ID NO: 191) |
| ZmFER1 | ZmFER1-F: | ZmFER1-R: CAACATCGCTGCCATAGTC |
| | CTTGCATCATTTCATGTCCCTAC (SEQ | (SEQ ID NO: 193) |
| | ID NO: 192) | |
| ZmTSB2 | ZmTSB2-F: GGTCGCACAAGCCCAACAC | ZmTSB2-R: |
| | (SEQ ID NO: 194) | CGAGATCAGAGGTGCCATTCC (SEQ |
| | | ID NO: 195) |
| ZmTSB1 | ZmTSB1-F1: CCTCCCACTCACGCTTTCC | ZmTSB1-R1: |
| | (SEQ ID NO: 196) | CACAGCATTGTTGATCTTGTG (SEQ |
| | | ID NO: 197) |
| ZmTSB1 | ZmTSB1-F2: CCCAGCCAGCCTATCTACC | ZmTSB1-R2: |
| | (SEQ ID NO: 198) | CTACCTGGAACTCGTCGTC (SEQ ID |
| | | NO: 199) |
| ZmIGS | ZmIGS-F: | ZmIGS-R: GGTCAGGATGCTCAGGCAC |
| | GAGGTGATCCAGTGGGAGAG (SEQ | (SEQ ID NO: 201) |
| | ID NO: 200) | |
| ZmAUX2 | ZmAUX2-F: | ZmAUX2-R: |
| | GCAGCATCACAAAGCAAACTG (SEQ | CCAAACTCCTCCTCTGACATC (SEQ |
| | ID NO: 202) | ID NO: 203) |
| ZmTAR2 | ZmTAR2-F: | ZmTAR2-R: |
| | GCATTACACCTTGGCACGTC (SEQ ID | GCACGGACAGCGGATCCTC (SEQ ID |
| | NO: 204) | NO: 205) |
| ZmYUC6 | ZmYUC6-F: GTACCTGCACCGCGACGAC | ZmYUC6-R: |
| | (SEQ ID NO: 206) | GTACCAGATCGAGCAGCAGG (SEQ |
| | | ID NO: 207) |

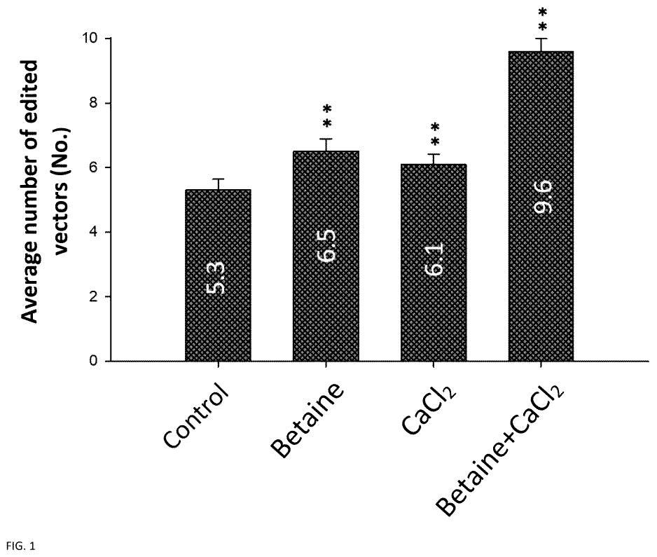

For each treatment, 30 gene-edited materials (120 in total) were randomly selected for editing type identification. For each plant, PCR amplification was performed using 23 pairs of primers, followed by Sanger sequencing to obtain accurate editing types. The number of edited loci was counted according to the following rule: each vector had two targets; if one target locus was edited (e.g., base deletion, insertion, substitution), if both target loci were edited, or if the fragment between the two targets was edited (e.g., fragment deletion, substitution), any of the above was considered that the vector functioned and an edit occurred and was counted as 1. Thus, the counted editing number refers to the number of vectors that functioned to edit in a single transformation event (FIG. 1).

### 5. Results and Phenotypic Selection

As shown in FIG. 1, regardless of whether betaine alone or calcium chloride alone was added, or both were added, the number of edits per single transformation event was significantly higher than that of the control, and the number of edited loci was the highest when both were added. According to statistics, compared with the control, betaine treatment alone increased editing efficiency by 22.6%, calcium chloride treatment alone increased editing efficiency by 15.1%, and betaine and calcium chloride synergistically promoted gene editing efficiency, increasing it by 81% over the control group. Therefore, betaine and calcium chloride can effectively promote the efficiency of aggregated editing of target loci and improve gene editing efficiency, providing a new approach for maize germplasm innovation.

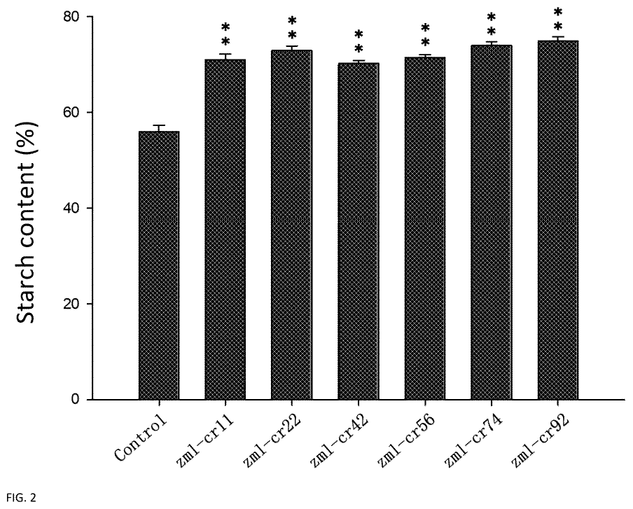

Seeds of homozygous gene-edited materials were planted in the field (Shandong) for multi-location and multi-replicate field phenotypic identification and screening. After pollination and seed harvesting, kernel starch content and mycotoxin content were measured; here, mycotoxin content is represented by the content of the well-known and important aflatoxin B1. Novel germplasm materials with good overall performance, such as high starch and low mycotoxin, were screened (FIG. 2 and Table 6). Six gene-edited materials with relatively high starch content were identified and screened, of which two were selected from transformation events obtained by betaine treatment, two from calcium chloride treatment, and two from betaine+calcium chloride treatment. Because Shandong is located in the Huang-Huai-Hai region, where climatic conditions are variable, an experimental site with relatively severe ear rot was selected from the multi-location replicated environments for measurement of kernel mycotoxin content. Seven germplasm with very low mycotoxin contents were selected therefrom (because contents below 3 μg/kg are difficult to measure, the value is expressed as <3 μg/kg) (Table 6), of which two were from transformation events obtained by betaine treatment, two from calcium chloride treatment, and three from combined betaine+calcium chloride treatment. Due to limitations in sample size, a sufficient number of gene-edited materials with both high starch and low mycotoxin contents have not yet been identified and screened, and thus it is not yet possible to statistically compare which treatment yields more germplasm with high starch and low mycotoxin contents. However, it can be confirmed that treatment with betaine, calcium chloride, or betaine+calcium chloride increased the average number of edits in a single transformation event, providing a new approach for efficiently, directionally, and precisely generating novel germplasm materials useful for breeding.

TABLE 6
Gene-Edited Materials Identified and Selected
with Lower Mycotoxin Contents

| Name | Mycotoxin Content (μg/kg) | Treatment |
|---|---|---|
| KN5585 | 6.2 | Control |
| zml-cr14 | <3 | Betaine 100 mM |
| zml-cr16 | <3 | Betaine 100 mM |
| zml-cr19 | <3 | Calcium chloride 50 mM |
| zml-cr44 | <3 | Calcium chloride 50 mM |
| zml-cr48 | <3 | Betaine 100 mM + Calcium |
| | | chloride 50 mM |
| zml-cr76 | <3 | Betaine 100 mM + Calcium |
| | | chloride 50 mM |
| zml-cr96 | <3 | Betaine 100 mM + Calcium |
| | | chloride 50 mM |

The above description is only of preferred embodiments of the present invention and is not intended to limit the present invention. Various changes and modifications may be made by one of ordinary skill in the art. Any modifications, equivalent substitutions, and improvements made within the spirit and principles of the present invention should fall within the scope of protection of the present invention.

1. A method for improving gene editing efficiency in maize, comprising: performing a genetic transformation using CRISPR/Cas9 multi-gene editing technology, wherein an infection medium used during the genetic transformation comprises 80-120 mM betaine and/or 30-60 mM calcium chloride.

2. The method for improving gene editing efficiency in maize of claim 1, comprising the following steps:
S1: selecting target genes and designing guide RNAs (gRNAs);
S2: using a CRISPR/Cas9 system to obtain dual-target knockout vectors by restriction enzyme digestion-ligation and genetic recombination, individually transforming the dual-target knockout vectors into Agrobacterium to obtain Agrobacterium cultures, and mixing the Agrobacterium cultures in equal volumes to prepare a pooled Agrobacterium culture;
S3: genetic transformation: transforming maize recipient material with the pooled Agrobacterium culture obtained in S2, and obtaining positive seedlings by herbicide-resistance selection; and
S4: planting and selecting homozygous edited plants, calculating the gene editing efficiency, and observing phenotypes.

3. The method for improving gene editing efficiency in maize of claim 2, wherein:
in step S1, based on maize genome sequence information, specific sgRNA sequences are designed for genes related to ear rot, mycotoxin and auxin content;
in step S2, the sgRNAs prepared in step S1 are integrated into a CRISPR/Cas9 vector, and after sequencing verification of correct construction, a plurality of dual-target knockout vectors are formed; and
the plurality of dual-target knockout vectors are individually transformed into Agrobacterium EHA105, and the Agrobacterium cultures are mixed in equal volumes to obtain the pooled Agrobacterium culture for subsequent genetic transformation.

4. The method for improving gene editing efficiency in maize of claim 3, wherein in step S2, the CRISPR/Cas9 vector is a binary expression vector.

5. The method for improving gene editing efficiency in maize of claim 4, wherein the binary expression vector is pCXB053.

6. The method for improving gene editing efficiency in maize of claim 2, wherein in step S3, the genetic transformation comprises the following steps:
S3-1: placing the maize embryos into 1.5-2 ml of a suspension and treating for 25-35 min;
S3-2: removing the suspension, adding the infection medium, and allowing infection to proceed statically for 3-8 min;
S3-3: transferring the immature maize embryos after infection to a co-cultivation medium and culturing in the dark at 22-24° C. for 3 days, and then transferring the immature maize embryos to a resting medium and culturing in the dark at 27-29° C. for 5-7 days;
S3-4: after completion of culture in S3-3, removing embryonic roots from the immature maize embryos and transferring the immature maize embryos to a first selection medium containing 5 mg/L bialaphos for 14 days, and then transferring the immature maize embryos to a second selection medium containing 8 mg/L bialaphos for 14 days to obtain resistant callus; and
S3-5: transferring the resistant callus to a differentiation medium, culturing at 24-26° C. under light at 5,000 lux for 3 weeks, and obtaining regenerated seedlings.

7. The method for improving gene editing efficiency in maize of claim 6, wherein the infection medium comprises ½ Murashige and Skoog (MS) medium, 68.5 g/L sucrose, 36 g/L glucose, 0.115 g/L L-proline, 200 mM acetosyringone, 200 mg/L cysteine, a mixed Agrobacterium suspension having an OD600 of 0.5-1, 100 mM betaine and 50 mM calcium chloride.
